(19) United States
(12) Patent Application Publication
(10) Pub. No.: US 2005/0240025 A1
(43) Pub. Date: Oct. 27, 2005

(54) Method for producing imidazolium salts

(75) Inventor: Frank Glorius, Leidenhofen (DE)

Correspondence Address: NORRIS, MCLAUGHLIN & MARCUS, PA, 875 THIRD STREET, 18TH FLOOR, NEW YORK, NY, 10022, US

(73) Assignee: Studiengesellschaft Kohle mbH, Mulheim an der Ruhr (DE)

(21) Appl. No.: 10/520,800

(22) PCT Filed: Jul. 8, 2003
(86) PCT No.: PCT/DE03/02285

§ 371 Date: Jan. 10, 2005

(30) Foreign Application Priority Data
Jul. 11, 2002 (DE) 102 31 368.7

(51) Int. Cl. C07D 498/04

(52) U.S. Cl. 548/218; 546/270.4

(57) ABSTRACT

The invention relates to a method for producing imidazolium salts by reacting bisimines or corresponding heterocycles with a combination consisting of an alkylating agent and of a metal salt serving as promoters of the reaction. This method makes it possible to obtain a multlitude of imidazolium salts under mild reaction conditions and in good yields. The imidazolium salts produced from heterocycles constitute novel compounds.

The invention relates to a process for preparing imidazolium salts by reacting bisimines or corresponding heterocycles with a combination of an alkylating agent and a metal salt as a promoter of the reaction. This process allows the preparation of a multitude of imidazolium salts under mild reaction conditions and in good yields. This synthetic process makes it possible to prepare new types of imidazolium salts of the general formulae II, IV and XI (especially chiral enantiomerically pure and highly substituted imidazolium salts), and also already known imidazolium salts of the general formula VI with an improved yield. The imidazolium salts may be converted by deprotonation to N-heterocyclic carbenes and transition metal complexes thereof. These complexes have a high thermal and chemical stability, and very good homogeneous catalyst properties for various reactions.

The use of N-heterocyclic carbenes of the imidazole type as ligands in homogeneous transition metal catalysis has become a significant field of research. Particularly processes for C—C—, C—O— and C—N-bond formation and applications in olefin metathesis have gained great significance. These include in particular successful applications in Heck, Suzuki, Sonogashira, Kumada and Stille cross-couplings, aryl aminations, α-arylations of amides, hydrosilylations, hydrogenations, 1,4-additions, hydroformylations, cyclopropanations of olefins, arylations and alkenylations of aldehydes, reductions of haloarenes, free-radical atom transfer polymerizations, olefin metathesis, ethylene/carbon monoxide copolymerizations, C—H activations and telomerizations of 1,3-dienes with alcohols. For example, DE 4447067 A1 describes cobalt and rhodium complexes having heterocyclic mono- or dicarbene ligands as catalysts for the industrially important hydroformylation. Industrial interest is also attracted by free-radical atom transfer polymerization, for which an iron(II)-carbene complex exhibits the currently highest polymerization rates observed in solvents with simultaneously low polydispersity. Additionally of particular significance are the numerous applications of ruthenium complexes of N-heterocyclic carbenes in olefin metathesis, which has clearly shown the advantages of N-heterocyclic carbene ligands over phosphane ligands, for example in DE 1981527.5.

The total sales of enantiomerically pure pharmaceuticals grew for the first time in the year 2000 to above 100 billion dollars, so that there is a great demand for enantiomerically pure substances. Transition metal complexes of chiral, enantiomerically pure N-heterocyclic carbenes are promising catalysts in asymmetric catalysis. (Comprehensive Asymmetric Catalysis; Eds.: E. N. Jacobsen, A. Pfaltz, H. Yamamoto; Springer: Berlin, 1999.) The first very successful applications of such chiral complexes in the hydrogenation of trisubstituted alkenes and in olefin metathesis confirm this potential. At the present time, there exist only relatively few chiral imidazolium salts. (See, for example, C. Bolm, M. Kesselgruber, G. Raabe, Organometallics (2002) 21, 707; J. J. Van Veldhuizen, S. B. Garber, J. S. Kingsbury, A. H. Hoveyda, J. Am. Chem. Soc. (2002) 124, 4954; T. J. Seiders, D. W. Ward, R. H. Grubbs, Org. Lett. (2001) 3, 3225; M. T. Powell, D.-R. Hou, M. C. Perry, X. Cui, K. Burgess, J. Am. Chem. Soc. (2001) 123, 8878; S. Lee, J. F. Hartwig, J. Org. Chem. (2001) 66, 3402; D. S. Clyne, J. Jin, E. Genest, J. C. Gallucci, T. V. RajanBabu, Org. Lett. (2000) 2, 1125.) The synthesis of novel, chiral imidazolium salts, especially imidazolium salts having a stereocenter directly adjacent, and the use of their transition metal complexes in asymmetric catalysis is therefore of great significance.

The deprotonation of imidazolium salts for the preparation of the corresponding N-heterocyclic carbenes and their transition metal complexes has found wide use as a method of choice. Generally usable synthetic methods for imidazolium salts are therefore of great interest. There already exist numerous synthetic methods for imidazolium salts. (Review: W. A. Herrmann, Angew. Chem. (2002) 114, 1342.) Starting from glyoxal, primary amines and formaldehyde, imidazolium salts can be formed under acidic reaction conditions (U.S. Pat. No. 5,182,405). Isolated bisimines obtained from glyoxal may likewise be reacted with acid and formaldehyde or with chloromethyl ethyl ether to give imidazolium salts (U.S. Pat. No. 5,077,414). Unsymmetric 1,3-disubstituted imidazolium salts may be obtained by alkylating monosubstituted imidazoles. The monosubstituted imidazoles required for this purpose may be obtained in analogy to the abovementioned synthesis from glyoxal, formaldehyde and a mixture of a primary amine with ammonium chloride. Saturated imidazolium salts may be obtained from substituted 1,2-bisamines by reaction with formaldehyde or trialkyl orthoformate.

Owing to their reaction conditions (usually in acidic medium, in the presence of nucleophiles and at relatively high temperature), the field of application of these synthetic methods is limited. The versatility of the substance classes suitable as a starting material is therefore restricted, so that numerous substitution patterns cannot be obtained with the known methods. In particular, it has hitherto not been possible to prepare many chiral imidazolium salts and imidazolium salts having acid-sensitive substituents. Among other compounds, the literature does not describe any examples of imidazolium salts of the general formulae II and IV. Furthermore, the above-described synthetic processes afford frequently only moderate yields after frequently long reaction times.

It has been found that, surprisingly, using a combination of alkylating agent and a metal salt as a promoter of the reaction, it is possible for the first time to prepare under mild conditions the imidazolium salts of the general formulae II, IV and XI shown below, and to prepare the imidazolium salts of the general formula VI in improved yield.

The invention provides a process for preparing imidazolium salts of the general formulae II, IV and VI, comprising the reaction of the corresponding substrates I, III and V, 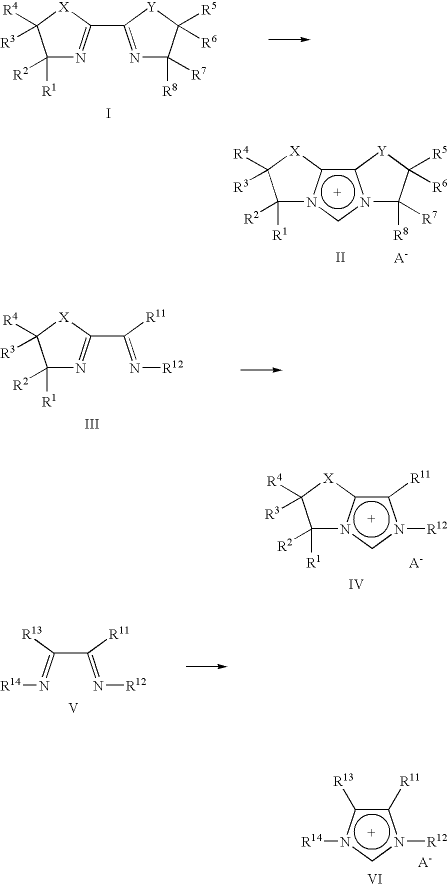
where
R¹, R², R³, R⁴, R⁵, R⁶, R⁷, R⁸, R¹¹, R¹², R¹³ and R¹⁴ are the same or different and are saturated or unsaturated, straight-chain, branched or cyclic, unsubstituted or substituted C₁₋₁₀-alkyl, C₂₋₅-alkenyl, C₂₋₅-alkynyl, C₇₋₁₉-aralkyl or C₆₋₁₄-aryl substituents, or R¹, R², R³, R⁴, R⁵, R⁶, R⁷, R⁸, R¹¹, R¹² and R¹³ may also be hydrogen or, together, form fused, substituted or unsubstituted substituents having 3-7 carbon atoms, R¹¹ and R¹³ may also be —OR¹⁶, —SR¹⁷ or —NR¹⁸R¹⁹, in which R¹⁶, R¹⁷, R¹⁸ and R¹⁹ may each be as defined for the R¹ to R⁸ and R¹¹ to R¹⁴ substituents, and R¹⁶, R¹⁷, R¹⁸, R¹⁹ and one of the R¹, R², R⁷, R⁸, R¹² and R¹⁴ substituents may also be a linker L to a further imidazolium salt of the formula II, IV or VI, X is O, S, an NR⁹ or CR^{9a}R^{9b} group in which R⁹, R^{9a} and R^{9b} are each hydrogen, saturated or unsaturated, straight-chain, branched or cyclic, unsubstituted or substituted C₁₋₁₀-alkyl, C₂₋₅-alkenyl, C₂₋₅-alkynyl, C₇₋₁₉-aralkyl or C₆₋₁₄-aryl substituents,
Y is O, S, an NR¹⁰ or NR^{10a}R^{10b} group in which R¹⁰, R^{10a}, R^{10b} are hydrogen, saturated or unsaturated, straight-chain, branched or cyclic, unsubstituted or substituted C₁₋₁₀-alkyl, C₂₋₅-alkenyl, C₂₋₅-alkynyl, C₇₋₁₉-aralkyl or C₆₋₁₄-aryl substituents, and
A⁻ is a mono- or polyvalent, organic or inorganic anion or a metal complex ion
   with a combination of an alkylating agent of the general formula VII, VIII or IX 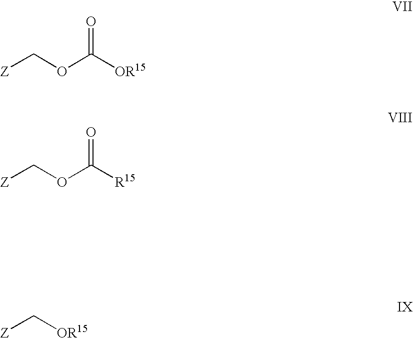
   where Z is a leaving group and R¹⁵ is as defined for R³, and a metal salt of the general formula
   MA
   where M is a mono- or polyvalent metal cation, a tetraorganoammonium compound or a triorganosilyl group, and A is as defined above for A⁻ as a promoter of the reaction.

The present invention further provides compounds of the general formulae II, IV and XI 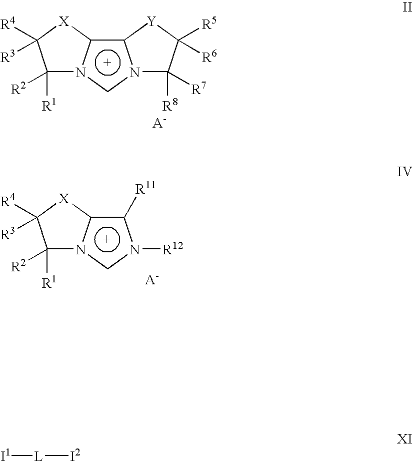
where
I¹ and I² are identical or different imidazolium salts of the formulae II, IV and VI which are joined to L at the position of the R¹, R², R⁷, R⁸, R¹² or R¹⁴ substituents, with the proviso that I¹ and I² are not both an imidazolium salt of the formulae VI,
the imidazolium salt of the formula VI, R¹, R², R³, R⁴, R⁵, R⁶, R⁷, R⁸, R¹¹, R¹², R¹³, R¹⁴, X, Y, L and A⁻ are each as defined above.

In one possible embodiment, R¹¹ and R¹² are joined together to form a substituted or unsubstituted cycle, preferably to form a pyridyl ring. Preferred substituents are C₁₋₆-alkyl and C₆₋₁₄-aryl. The substituents are preferably bonded to the carbon adjacent to the ring nitrogen.

In the above-defined compounds, it is preferred that R¹, R², R³, R⁴, R⁵, R⁶, R⁷R⁸, R⁹, R¹⁰, R^{9a}, R^{9b}, R^{10a}, R^{10b}, R¹¹, R¹²R¹³, R¹⁴, R¹⁵, R¹⁶, R¹⁷, R¹⁸, R¹⁹, are the same or different and are each saturated or unsaturated, straight-chain, branched or cyclic, unsubstituted or substituted C₁₋₆-alkyl, C₂₋₄-alkenyl, C₂₋₄-alkynyl, C₇₋₁₀-aralkyl or phenyl groups.

The C₁₋₆-alkyl substituents may be selected from methyl, ethyl, n-propyl, i-propyl, n-butyl, i-butyl, t-butyl, n-pentyl, i-pentyl, t-pentyl, n-hexyl, i-hexyl, t-hexyl, cyclopropyl, cyclobutyl, cyclopentyl or cyclohexyl. The C₂₋₄-alkenyl substituents may be selected from ethenyl, propenyl or butenyl, the C₂₋₄-alkynyl substituents from ethynyl, propynyl or butynyl. The C₇₋₁₀-aralkyl substituents may be selected from benzyl, phenylethyl, phenylpropyl and phenylbutyl.

The R¹ to R¹⁹ substituents may be substituted by one or more, identical or different amine, nitro, nitrile, isonitrile, ether, alcohol, aldehyde or ketone groups, carboxylic acid derivatives, in particular esters or amides, halogenated, in particular fluorinated or perfluorinated, hydrocarbon substituents, carbohydrate, phosphane, phosphane oxide, phosphane sulfide, phosphole groups, phosphite derivatives, aliphatic or aromatic sulfonic acid derivatives, the salts, esters or amides thereof, silyl functions, boryl groups or heterocyclic substituents. Further suitable substituents, especially when they are aromatic systems, are C₁-C₆-alkyl, C₂-C₄-alkenyl or C₂-C₄-alkynyl groups. In a particularly preferred embodiment, one of the R¹, R², R⁷, R⁸, R¹² and R¹⁴ groups is substituted by an azolium salt or a pyridine ring.

In a further embodiment, one of the R¹, R², R⁷, R⁸, R¹² and R¹⁴ substituents is a linker L to a further imidazolium salt of the formula II, IV or VI. L may in particular be a C₁₋₄-alkylene group, (e.g. a methylene, ethylene, propylene or butylene group), a C₅₋₁₂-cycloalkylene group (e.g. a 1,2- or 1,4-cyclohexylene group), a C₆₋₁₂-arylene group (e.g. a 1,2-, 1,3- or 1,4-phenylene group) or a C₆₋₁₂-heteroarylene group (e.g. a 2,3-, 2,4- or 2,6-pyridinylene group). The aforementioned groups may optionally be substituted (for example by C₁₋₄-alkyl groups, C₁₋₄-alkoxy groups, halogen atoms, hydroxyl groups, etc.) or be interrupted by a heteroatom (e.g. O or NH) or a cyclic groups (e.g. a phenyl or cyclohexyl groups). Particular preference is given to an imidazolium salt which has the general formula X 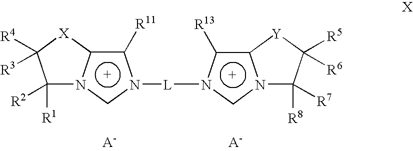
where the variables are each as defined above.

It is also preferred that the mono- or polyvalent organic anion A⁻ in the general formulae II, IV, VI and XI is a sulfate, halide, pseudohalide, borate, phosphate or metal complex ion or an optionally halogenated sulfonate, carboxylate or acetylacetonate ion, and A⁻ is in particular a triflate, mesylate, tosylate, nonaflate, tresylate, benzenesulfonate, brosylate, nosylate, fluorosulfonate, tetraphenylborate, tetrakis[3,5-bis(trifluoromethyl)phenyl]borate (BARF), tetrafluoroborate, hexafluorophosphate, hexafluoro-antimonate, acetate, trifluoroacetate, perchlorate, tetracarbonylcobaltate or hexafluoroferrate(III) ion. The particularly preferred anion among the anions A⁻ mentioned is the triflate ion.

The process (1) includes the use of an alkylating agent of the above-defined general formula VII, VIII or IX. The leaving group Z in this alkylating agent is preferably a halide, pseudohalide or (optionally halogenated) carboxylate, more preferably a halide, more preferably a chloride. Preferred alkylating agents are those in which R¹⁵ is an unsubstituted or substituted phenyl, benzyl or C₁₋₄-alkyl substituents which may in each case contain one or more substituents, in particular ether, ester or silyl substituents. Particular preference is given to chloromethyl pivalate, chloromethyl butyrate, chloromethyl ethyl ether, (2-methoxyethoxy)methyl chloride and (2-chloro-methoxyethyl)trimethylsilane.

Preferred metal salts of the general formula MA which can be used in the process (1) are those in which the mono- or polyvalent metal cation M is a silver(I), alkali metal and alkaline earth metal, lanthanide, lead(II), mercury(II), cadmium(II), thallium(I), copper(II), zinc(II) or aluminum(III) ion, and those in which the tetraorganoammonium compound is a tetraalkylammonium compound and finally those in which the triorganosilyl group is a trialkylsilyl group. Particularly preferred metal salts are those in which M is silver(I) and A is a sulfonate, sulfate, halide, pseudohalide, oxide, borate, phosphate, carboxylate, acetylacetonate or metal complex ion, preferably a trifluoromethanesulfonate (triflate), methanesulfonate(mesylate), p-toluenesulfonate (tosylate), nonafluorobutanesulfonate (nonaflate), 2,2,2-trifluoroethane-sulfonate (tresylate), benzenesulfonate, p-bromo-benzenesulfonate (brosylate), p-nitrobenzenesulfonate (nosylate), fluorosulfonate, tetraphenylborate, tetrakis[3,5-bis(trifluoromethyl)phenyl]borate (BARF), tetrafluoroborate, hexafluorophosphate, hexafluoro-antimonate, acetate, trifluoroacetate, perchlorate, tetracarbonylcobaltate or hexafluoroferrate(III) ion, and is more preferably a triflate ion.

Typically, alkylating agent and metal salt are used in at least stoichiometric amount, preferably in a from 5 to 100% excess in relation to the substrate. The ratio of alkylating agent to metal salt may be varied within a wide range and is preferably from 2:1 to 1:2, more preferably from 1.2:1 to 1:1.2.

The imidazolium salts of the general formulae II, IV and VI are synthesized preferably with the exclusion of air and moisture. It has been found to be particularly advantageous to add the alkylating agent to a solution of the appropriate starting material of the general formula I, III or V and the metal salt in an organic solvent. Suitable solvents may be acetone, tetrahydrofuran, diethyl ether, methyl tert-butyl ether, 1,2-dimethoxyethane, 1,4-dioxane, petroleum ether, dimethyl sulfoxide, N,N-dimethylformamide, 1-methyl-2-pyrrolidone, 1,3-dimethyl-3,4,5,6-tetrahydro-2(1H)-pyrimidone, acetonitrile, propionitrile, ethyl acetate, benzene, toluene, xylene, benzine, chloroform, 1,2-dichloroethane and methylene chloride, preferably methylene chloride. After stirring at from −78 to 120° C., preferably at from 0 to 70° C., in particular at from 20 to 50° C., for a few hours, the reaction solution is purified in a conventional manner depending on the physical properties of the products, for example by column chromatography or crystallization.

The inventive imidazolium salts of the present invention are in particular those compounds in which R¹, R², R³, R⁴, R⁵, R⁶, R⁷, R⁸, R¹¹, R¹², R¹³, R¹⁴, R¹⁵, R¹⁶, R¹⁷, R¹⁸, X, Y, L and A⁻ each have the preferred definition specified above.

Particular preference is given to compounds having the following structural formulae: 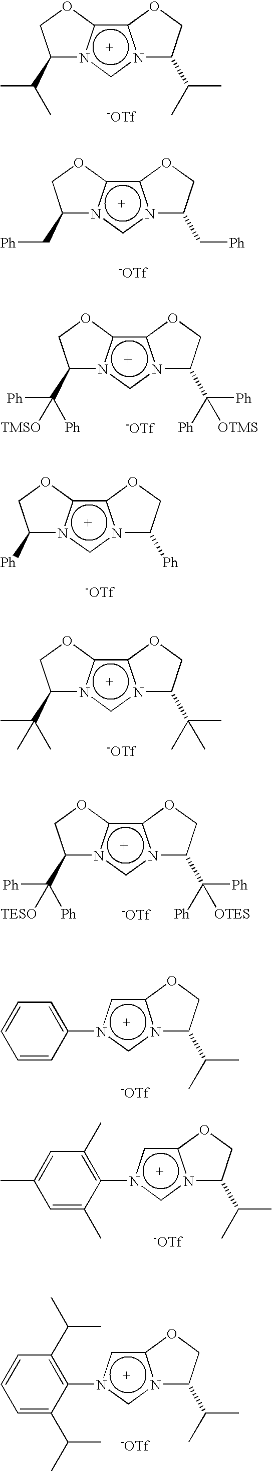 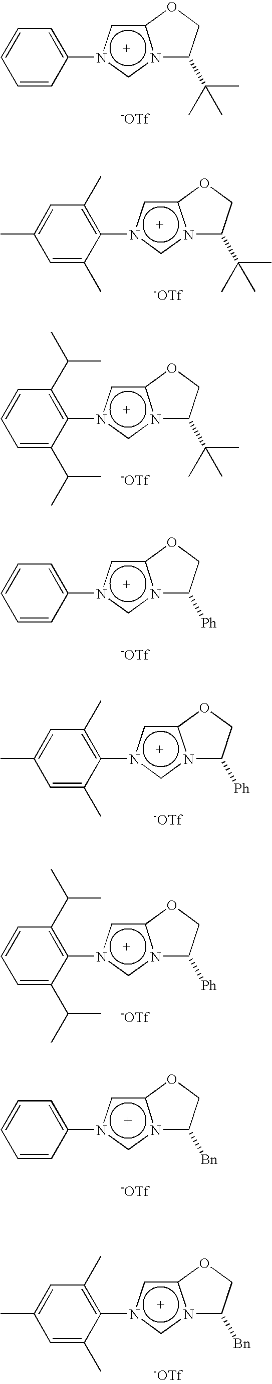 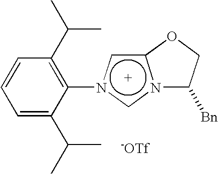
where OTf is trifluoromethanesulfonate (triflate), Ph is phenyl, TMS is trimethylsilyl, TES is triethylsilyl and Bn is benzyl.

Preference is further given to the following compounds of the formula X 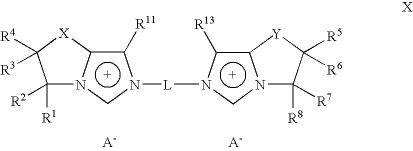
where each variable is as defined above. Among these, preference is given particularly to compounds having the following structural formulae: 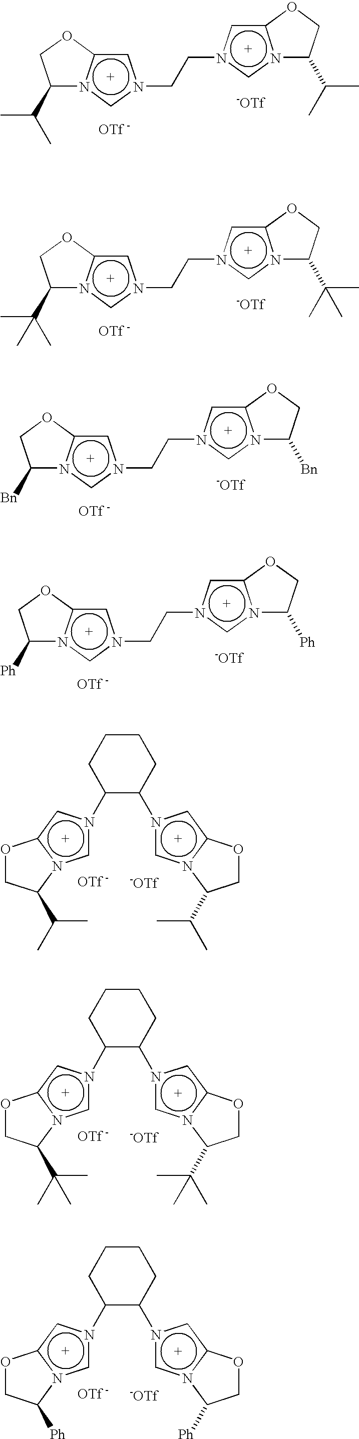 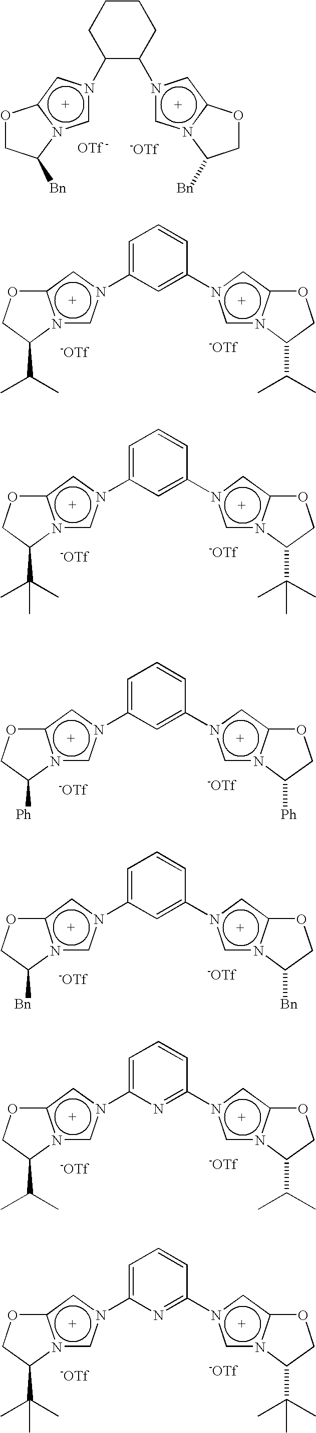 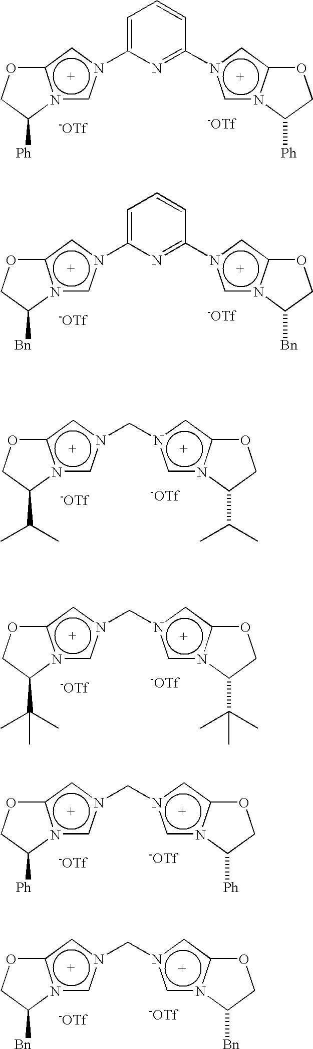
where OTf is trifluoromethanesulfonate (triflate), Ph is phenyl and Bn is benzyl.

The compounds mentioned specifically above may likewise have tetrafluoroborate, mesylate, tosylate, nonaflate or hexafluoroantimonate instead of triflate as the counteranion.

The process according to the invention enables the preparation of a multitude of hitherto unknown, achiral and chiral imidazolium salts in surprisingly good yield, high purity and, if appropriate, high optical purity. This can be attributed firstly to the wide structural variety of the starting materials of the general formulae I, III and V and secondly to the mild alkylation conditions which surprisingly become possible as a result of the combined use of an alkylating agent and a metal salt as a promoter. The process according to the invention has therefore been found to be particularly useful for preparing imidazolium salts from acid-sensitive substrates and for the preparation of chiral imidazolium salts. In addition, the process can be used to synthesize both milligram and multigram amounts of imidazolium salts. Owing to the simple reaction, the process is also suitable for industrial application.

The imidazolium salts, preparable by this process, of the general formulae II, IV and VI can be deprotonated in accordance with the literature and thus converted to N-heterocyclic carbenes or transition metal complexes thereof. (Review: W. A. Herrmann, Angew. Chem. (2002) 114, 1342; A. J. Arduengo, III, Acc. Chem. Res. (1999) 32, 913.) These transition metal-carbene complexes may be used as catalysts in homogeneous catalysis, and chiral, enantiomerically pure imidazolium salts of the general formulae II, IV and VI lead to chiral transition metal complexes which can be used in particular in asymmetric catalysis. Especially the novel imidazolium salts of the general formulae II and IV in which the imidazolium ring is bridged by one ring (IV) or two rings (II) are promising in this context. Suitable substitution of these bridges with the R¹, R², R³, R⁴, R⁵, R⁶, R⁷, and R⁸ substituents allows chiral, enantiomerically pure imidazolium salts with rigid geometry to be obtained, whose transition metal-carbene complexes may find use in asymmetric catalysis.

### WORKING EXAMPLES

Conversion of bioxazolines 1 to the corresponding imidazolium triflates 2 according to the following equation.

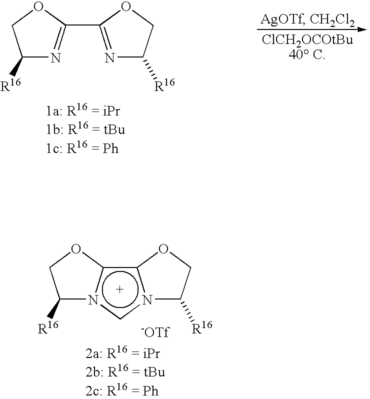

### Example 1

### Preparation of imidazolium triflate 2a

In a flame dried and argonized Schlenk vessel, with exclusion of light, air and moisture, chloromethyl pivalate (4.6 ml, 31.2 mmol) was added to a solution of bioxazoline 1a (5.0 g, 22.2 mmol) and silver triflate (6.8 g, 26.6 mmol) in methylene chloride (75 ml) and the reaction vessel was sealed. After stirring with exclusion of light at 40° C. for 24 hours, the reaction mixture cooled to room temperature was filtered through a glass frit, and the filter residue was washed with methylene chloride (25 ml) and the filtrate concentrated. Purification of the residue by column chromatography (4×10 cm, 20:1 CH₂Cl₂/MeOH) and subsequent recrystallization from THF (30 ml), toluene (150 ml) and pentane (50 ml) gave the imidazolium triflate 2a (6.85 g, 80%).

[a]²⁰_{D}=+55.0 (c 1.0, CH₂Cl₂); ¹H NMR (400 MHz, CDCl₃) δ 8.73 (s, 1H, NCHN), 5.07 (dd, J=7.9, 9.0 Hz, 2H, CH₂), 4.98-4.93 (m, 2H, CHCH₂), 4.83 (dd, J=4.1, 9.0 Hz, 2H, CH₂), 2.33 (m, 2H, CHCH₃), 1.03 (d, J=6.9 Hz, 6H, CH₃), 0.99 (d, J=6.9 Hz, 6H, CH₃), ¹³C NMR (100 MHz, CDCl₃) δ 125.6 (NCO), 120.6 (q, J=320 Hz, CF₃), 116.3 (NCHN), 79.1 (CH₂), 63.9 (CHCH₂), 31.1 (CHCH₃), 17.6 (CH₃), 16.7 (CH₃), ¹⁹F NMR (300 MHz, CDCl₃) δ −78.7 (CF₃).

### Example 2

### Preparation of imidazolium triflate 2b

In a flame dried and argonized Schlenk vessel, with exclusion of light, air and moisture, chloromethyl pivalate (0.35 ml, 2.4 mmol) was added to a solution of bioxazoline 1b (425 mg, 1.7 mmol) and silver triflate (518 mg, 2.0 mmol) in methylene chloride (15 ml) and the reaction vessel was sealed. After stirring with exclusion of light at 40° C. for 8 hours, the reaction mixture cooled to room temperature was filtered through a glass frit, and the filter residue was washed with methylene chloride (10 ml) and the filtrate concentrated. Purification of the residue by column chromatography (2.5×10 cm, 20:1 CH₂Cl₂/MeOH) and subsequent recrystallization from THF (5 ml), toluene (20 ml) and pentane (5 ml) gave the imidazolium triflate 2b (521 mg, 75%).

[a]²_{D}=+69.5 (c 1.0, CH₂Cl₂); ¹H NMR (400 MHz, CDCl₃) δ 8.61 (s, 1H, NCHN), 5.07 (dd, J=7.9, 9.3 Hz, 2H, CH₂), 4.92 (dd, J=3.3, 9.4 Hz, 2H, CH₂), 4.83 (dd, J=3.2, 7.8 Hz, 2H, CH), 1.08 (s, 18H, CH₃); ¹³C NMR (75 MHz, CDCl₃) δ 125.9 (NCO), 120.6 (q, J=320 Hz, CF₃), 117.0 (NCH), 78.8 (CH₂), 68.2 (CH), 34.1 (CCH₃), 25.3 (CH₃); ¹⁹F NMR (300 MHz, CDCl₃) 8-78.6 (CF₃).

### Example 3

### Preparation of imidazolium triflate 2c

In a flame dried and argonized Schlenk vessel, with exclusion of light, air and moisture, chloromethyl pivalate (0.85 ml, 5.8 mmol) was added to a solution of bioxazoline 1c (1.2 g, 4.1 mmol) and silver triflate (2.6 g, 10.3 mmol) in methylene chloride (20 ml) and the reaction vessel was sealed. After stirring with exclusion of light at 40° C. for 15 hours, the reaction mixture cooled to room temperature was filtered through a glass frit, and the filter residue was washed with methylene chloride (10 ml) and the filtrate concentrated. Purification of the residue by double column chromatography (3×10 cm, 20:1 CH₂Cl₂/MeOH) gave the imidazolium triflate 2c (430 mg, 23%).

[a]²⁰_{D}=+226.3 (c 0.8, CH₂Cl₂); ¹H NMR (400 MHz, CDCl₃) δ 7.72 (s, 1H, NCHN), 7.42-7.31 (m, 10H, CHₐᵣ), 6.05 (t, J=7.2 Hz, 2H, CHPh), 5.41 (dd, J=7.9, 8.9 Hz, 2H, CH₂), 4.90 (dd, J=6.6, 9.0 Hz, 2H, CH₂); ¹³C NMR (100 MHz, CDCl₃) δ 133.4 (C), 130.3 (CH) 129.8 (CH), 127.1 (CH), 126.6 (NCO), 120.6 (q, J=320 Hz, CF₃), 114.9 (NCHN), 84.1 (CH₂), 62.2 (CHPh); ¹⁹F NMR (300 MHz, CDCl₃) δ −78.6 (CF₃).

Conversion of imine oxazolines 3 to the corresponding imidazolium triflates 4 according to the following equation

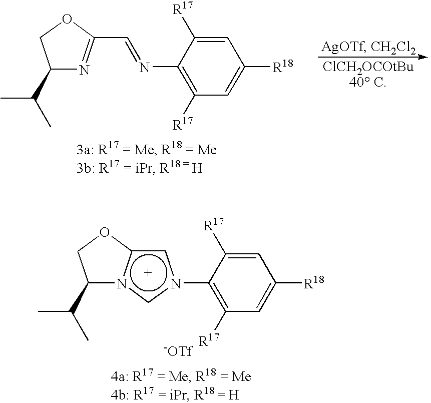

### Example 4

### Preparation of imidazolium triflate 4a

In a flame dried and argonized Schlenk vessel, with exclusion of light, air and moisture, chloromethyl pivalate (0.27 ml, 1.8 mmol) was added to a solution of imine oxazoline 3a (330 mg, 1.3 mmol) and silver triflate (395 mg, 1.5 mmol) in methylene chloride (10 ml) and the reaction vessel was sealed. After stirring with exclusion of light at 40° C. for 16 hours, the reaction mixture cooled to room temperature was filtered through a glass frit, and the filter residue was washed with methylene chloride (10 ml) and the filtrate concentrated. Purification of the residue by double column chromatography (2×10 cm, CH₂Cl₂ to 15:1 CH₂Cl₂/MeOH) gave the imidazolium triflate 4a (432 mg, 80%).

[a]²⁰_{D}=+19.5 (c 0.9, CHCl₃); ¹H NMR (400 MHz, CDCl₃) δ 8.81 (d, J=1.4 Hz, 1H, NCHN), 7.02 (s, 1H, CHₐᵣ), 7.00 (s, 1H, CHₐᵣ), 6.29 (d, J=1.4 Hz, NCHC), 5.42-5.39 (m, 1H, CHCH₂), 5.29 (t, J=8.7 Hz, 1H, CH₂), 4.99 (dd, J=3.7, 9.2 Hz, 1H, CH₂), 2.54-2.47 (m, 1H, CHCH₃), 2.35 (s, 3H, CH₃), 2.16 (s, 3H, CH₃), 2.07 (s, 3H, CH₃), 1.04 (d, J=6.9 Hz, 3H, CHCH₃), 0.98 (d, J=6.9 Hz, 3H, CHCH₃); ¹³C NMR (100 MHz, CDCl₃) δ 151.3 (C), 141.4 (C), 135.0 (C), 133.8 (C), 131.5 (C), 130.0 (CHₐᵣ), 129.5 (CHₐᵣ), 127.8 (NCHN), 120.6 (q, J=320 Hz, CF₃), 95.4 (NCHC), 79.6 (CH₂), 63.1 (CHCH₂), 31.1 (CHCH₃), 21.1 (CH₃), 17.4 (CH₃), 17.2 (CH₃), 17.0 (CH₃), 16.2 (CH₃); ¹⁹F NMR (300 MHz, CDCl₃) δ −78.7 (CF₃).

### Example 5

### Preparation of imidazolium triflate 4b

In a flame dried and argonized Schlenk vessel, with exclusion of light, air and moisture, chloromethyl pivalate (0.42 ml, 2.8 mmol) was added to a solution of imine oxazoline 3b (600 mg, 2.0 mmol) and silver triflate (617 mg, 2.4 mmol) in methylene chloride (17 ml) and the reaction vessel was sealed. After stirring with exclusion of light at 40° C. for 16 hours, the reaction mixture cooled to room temperature was filtered through a glass frit, and the filter residue was washed with methylene chloride (10 ml) and the filtrate concentrated. Purification of the residue by double column chromatography (2.5×10 cm, CH₂Cl₂ to 15:1 CH₂Cl₂/MeOH) gave the imidazolium triflate 4b (771 mg, 83%).

[a]²⁰_{D}=+23.8 (c 0.9, CHCl₃); ¹H NMR (400 MHz, CDCl₃) δ 8.86 (d, J=1.5 Hz, 1H, NCHN), 7.55-7.51 (m, 1H, CHₐᵣ), 7.33-7.27 (m, 2H, CHₐᵣ), 6.33 (d, J=1.5 Hz, NCHC), 5.47-5.43 (m, 1H, CHCH₂), 5.33 (t, J=8.7 Hz, 1H, CH₂), 5.02 (dd, J=3.6, 9.2 Hz, 1H, CH₂), 2.58-2.51 (m, 2H, CHCH₃), 2.32 (sept, J=6.9 Hz, 1H, CHCH₃), 1.26 (d, J=6.8 Hz, 3H, CH₃), 1.21 (d, J=7.0 Hz, 3H, CH₃), 1.19 (d, J=7.0 Hz, 3H, CH₃), 1.17 (d, J=6.8 Hz, 3H, CH₃), 1.04 (d, J=6.9 Hz, 3H, CH₃), 0.98 (d, J=6.9 Hz, 3H, CH₃); ¹³C NMR (100 MHz, CDCl₃) δ 151.2 (C), 146.1 (C), 145.0 (C), 132.0 (CHₐᵣ), 130.9 (C), 128.4 (NCHN), 125.0 (CHₐᵣ), 124.3 (CHₐᵣ), 120.6 (q, J=320 Hz, CF₃), 96.7 (NCHC), 79.7 (CH₂), 63.2 (CHCH₂), 31.2 (CH), 28.8 (CH), 28.5 (CH), 24.5 (CH₃), 24.5 (CH₃), 23.9 (CH₃), 23.8 (CH₃), 17.3 (CH₃), 16.1 (CH₃); ¹⁹F NMR (300 MHz, CDCl₃) δ −78.7 (CF₃).

Conversion of bisimines 5 to the corresponding imidazolium triflates 6 according to the following equations

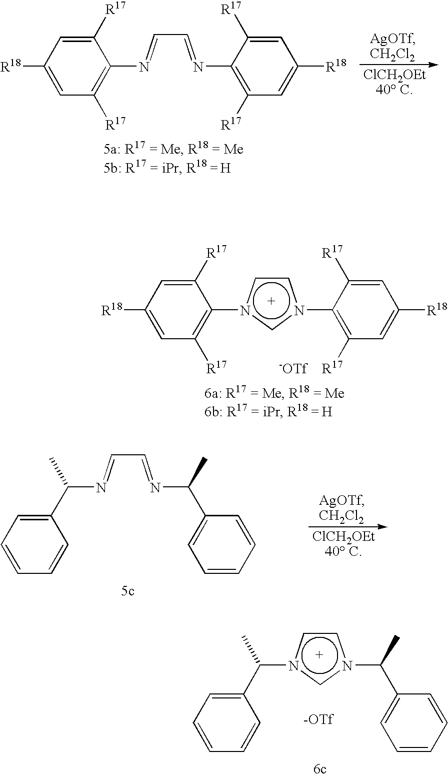

### Example 6

### Preparation of 1,3-bis(2,4,6-trimethylphenyl)imidazolium triflate (6a)

In a flame dried and argonized Schlenk vessel, with the exclusion of light, air and moisture, chloromethyl ethyl ether (0.047 ml, 0.48 mmol) was added to a solution of bisimine 5a (100 mg, 0.34 mmol) and silver triflate (105 mg, 0.41 mmol) in methylene chloride (2 ml), and the reaction vessel was sealed. After stirring with the exclusion of light at 40° C. for 16 hours, the reaction mixture cooled to room temperature was filtered through a glass frit, the filter residue was washed with methylene chloride (2 ml) and the filtrate was concentrated. Purification of the residue by recrystallization from toluene gave the imidazolium triflate 6a (129 mg, 83%).

¹H NMR (400 MHz, CDCl₃) δ 9.20 (t, J=1.4 Hz, 1H, NCHN), 7.56 (d, J=1.4 Hz, 2H, NCHCHN), 7.03 (s, 4H, CHₐᵣ) 2.35 (s, 6H, p-CH₃), 2.11 (s, 12H, o-CH₃; ¹³C NMR (100 MHz, CDCl₃) δ 141.5 (C), 137.9 (NCHN), 134.0 (C), 130.4 (C), 129.9 (CHₐᵣ), 124.9 (NCHCHN), 120.4 (q, J=321 Hz, CF₃), 21.1 (p-CH₃), 17.2 (o-CH₃); ¹⁹F NMR (300 MHz, CDCl₃) δ −78.9 (CF₃).

### Example 7

### Preparation of 1,3-bis(2,6-diisopropylphenyl)imidazolium triflate (6b)

In a flame dried and argonized Schlenk vessel, with the exclusion of light, air and moisture, chloromethyl ethyl ether (0.036 ml, 0.37 mmol) was added to a solution of bisimine 5b (100 mg, 0.27 mmol) and silver triflate (82 mg, 0.32 mmol) in methylene chloride (1.5 ml), and the reaction vessel was sealed. After stirring with the exclusion of light at 40° C. for 1 hour, the reaction mixture cooled to room temperature was filtered through a glass frit, the filter residue was washed with methylene chloride (2 ml) and the filtrate was concentrated. Purification of the residue by recrystallization from toluene gave the imidazolium triflate 6b (116 mg, 81%).

¹H NMR (400 MHz, CDCl₃) δ 9.13 (t, J=1.6 Hz, 1H, NCHN), 7.79 (d, J=1.6 Hz, 2H, NCHCHN), 7.57 (t, J=7.9 Hz, 2H, CHₐᵣ), 7.34 (d, J=7.9 Hz, 4H, CHₐᵣ), 2.40 (sept, J=6.8 Hz, 2H, CH), 1.26 (d, J=6.8 Hz, 6H, CH₃), 1.20 (d, J=6.8 Hz, 6H, CH₃); ¹³C NMR (100 MHz, CDCl₃) δ 144.9 (C), 138.2 (NCHN), 132.1 (CHₐᵣ), 129.7 (C), 126.3 (NCHCHN), 124.7 (CHₐᵣ), 120.5 (q, J=321 Hz, CF₃), 29.1 (CH), 24.2 (CH₃), 23.8 (CH₃); ¹⁹F NMR (300 MHz, CDCl₃) δ −78.9 (CF₃).

### Example 8

### Preparation of (S,S)-1,3-bis(1-phenylethyl)imidazolium triflate (6c)

In a flame dried and argonized Schlenk vessel, with the exclusion of light, air and moisture, chloromethyl ethyl ether (1.4 ml, 14.0 mmol) was added at 0° C. to a solution of bisimine 5c (2.6 g, 10.0 mmol) and silver triflate (3.1 g, 12.0 mmol) in methylene chloride (20 ml), and the reaction vessel was sealed. After stirring with the exclusion of light at 40° C. for 1 hour, the reaction mixture cooled to room temperature was filtered through a glass frit, the filter residue was washed with methylene chloride (20 ml) and the filtrate was concentrated. Purification of the residue by column chromatography (3.5×12 cm, CH₂Cl₂ to 15:1 CH₂Cl₂/MeOH) gave the imidazolium triflate 6c (3.5 g, 81%).

[a]²⁰_{D}=−21.5 (c 1.1, CHCl₃); ¹H NMR (400 MHz, CDCl₃) δ 9.51 (t, J=1.7 Hz, 1H, NCHN), 7.41-7.35 (m, 10H, CHₐᵣ), 7.20 (d, J=1.7 Hz, 2H, NCHCHN), 5.77 (q, J=7.0 Hz, 2H, CH), 1.95 (d, J=7.0 Hz, 6H, CH₃); ¹³C NMR (100 MHz, CDCl₃) δ 137.6 (C), 134.6 (NCHN), 129.4 (CHₐᵣ, CHₐᵣ), 126.9 (CHₐᵣ), 120.8 (NCHCHN), 120.7 (q, J=320 Hz, CF₃), 60.2 (CH), 20.8 (CH₃); ¹⁹F NMR (300 MHz, CDCl₃) δ −78.5 (CF₃).

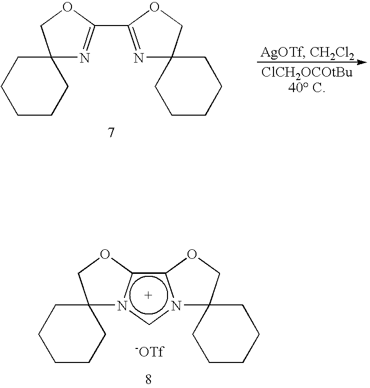

### Example 9

### Preparation of imidazolium triflate 8

In a Schlenk vessel, with the exclusion of light, air and moisture, chloromethyl pivalate (1.25 ml, 8.4 mmol) was added to a suspension of silver triflate (2.2 g, 8.4 mmol) in methylene chloride (30 ml) and the reaction solution was stirred for 45 minutes. After the solid formed had settled, the solution was introduced with a syringe into a second reaction vessel in which was disposed the bioxazoline 7 (1.6 g, 5.8 mmol). The solution was stirred at 40° C. for 20 hours and the reaction mixture cooled to room temperature was subsequently concentrated under reduced pressure. Purification of the residue by column chromatography (2.5×10 cm, 20:1 to 10:1 CH₂Cl₂/MeOH) and subsequent recrystallization from a mixture of THF (10 ml), toluene (40 ml) and pentane (40 ml) gave the imidazolium triflate 8 (2.2 g, 85%) in the form of colorless crystals.

¹H NMR (400 MHz, CDCl₃) δ 9.12 (s, 1H, NCHN), 4.80 (s, 4H, OCH₂), 2.32 (td, J=3.8, 12.5 Hz, 4H, CH₂), 2.10-1.98 (m, 8H, CH₂), 1.74-1.58 (m, 4H, CH₂), 1.46-1.37 (m, 4H, CH₂); ¹³C NMR (100 MHz, CDCl₃) δ 124.6 (NCO), 120.8 (q, J=319 Hz, CF₃), 113.9 (NCHN), 85.6 (OCH₂), 67.5 (CCH₂), 34.7 (CH₂), 23.5 (CH₂), 23.1 (CH₂); ¹⁹F NMR (300 MHz, CDCl₃) δ −78.5 (CF₃).

The examples 10 to 13 were carried out in substantial analogy to the method specified for example 9:

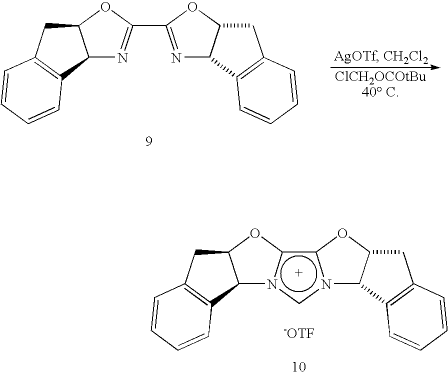

### Example 10

### Preparation of imidazolium triflate 10

3.4 g, 70%, colorless crystals; [a]²⁰_{D}=+184.8 (c 0.9, CH₂Cl₂); ¹H NMR (400 MHz, CDCl₃) δ 9.48 (S, 1H, NCHN), 7.83 (d, J=7.2 Hz, 2H, CHₐᵣ), 7.40-7.28 (m, 6H, CHₐᵣ), 6.32 (d, J=6.4 Hz, 2H, CHN), 6.11-6.09 (m, 2H, CHO), 3.56-3.44 (m, 4H, CH₂); ¹³C NMR (100 MHz, CDCl₃) δ 139.6 (C), 134.8 (C), 130.8 (CH), 129.0 (CH), 126.1 (CH), 125.3 (CH), 124.9 (C), 120.7 (q, J=320 Hz, CF₃), 114.4 (NCHN), 95.1 (OCH), 66.9 (NCHC), 38.3 (CH₂); ¹⁹F NMR (300 MHz, CDCl₃) δ −78.6 (CF₃).

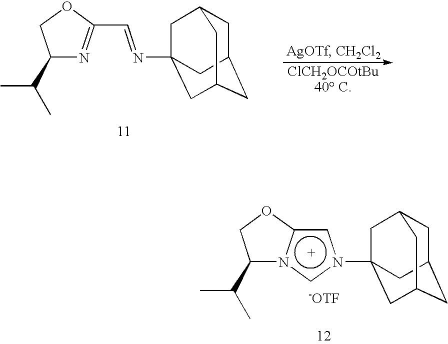

### Example 11

### Preparation of imidazolium triflate 12

1.2 g, 56%, white foam; [a]²⁰_{D}=+25.9 (c 1.1, CH₂Cl₂); ¹H NMR (400 MHz, CDCl₃) δ 8.94 (d, J=1.7 Hz, NCHN), 6.49 (d, J=1.7 Hz, CH), 5.15-5.08 (m, 2H, CH & CH₂), 4.92-4.86 (m, 1H, CH₂), 2.47-2.38 (m, 1H, CH), 2.30 (S, 3H, CH), 2.13 (d, J=2.9 Hz, 6H, CH₂), 1.78-1.76 (m, 6H, CH₂), 1.02 (d, J=6.9 Hz, 3H, CH₃), 0.91 (d, J=6.8 Hz, 3H, CH₃); ¹³C NMR (100 MHz, CDCl₃) δ 151.1 (C), 124.5 (CH), 120.7 (q, J=322 Hz, CF₃), 90.6 (CH), 79.2 (CH₂), 62.9 (CH), 61.3 (C), 42.6 (CH₂), 35.3 (CH₂), 30.9 (CH), 29.4 (CH), 17.8 (CH₃), 16.2 (CH₃); ¹⁹F NMR (300 MHz, CDCl₃) δ −78.6 (CF₃).

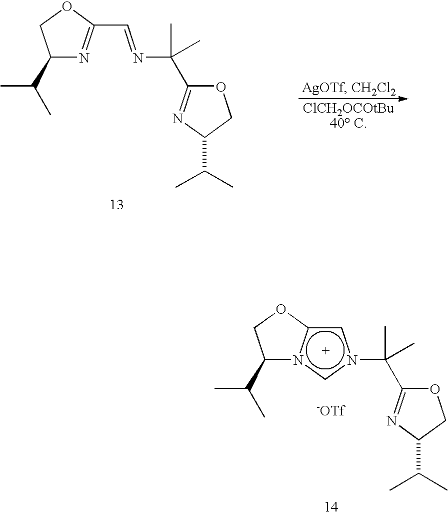

### Example 12

### Preparation of imidazolium triflate 14

0.7 g, 56%, colorless oil; [a]²⁰_{D}=−21.3 (c 1.1, CH₂Cl₂); ¹H NMR (400 MHz, CDCl₃) δ 9.09 (d, J=1.6 Hz, 1H, NCHN), 6.46 (d, J=1.6 Hz, 1H, CH), 5.17-5.13 (m, 2H), 4.94-4.90 (m, 1H), 4.36 (dd, J=8.6, 9.7 Hz, 1H), 4.06 (t, J=8.2 Hz, 1H), 3.98-3.92 (m, 1H), 2.47-2.43 (m, 1H), 1.96 (s, 3H), 1.90 (s, 3H), 1.78-1.71 (m, 1H), 1.03 (d, J=6.9 Hz, 3H), 0.92 (d, J=6.8 Hz, 3H), 0.91 (d, J=6.7 Hz, 3H), 0.85 (d, J=6.8 Hz, 3H); ¹³C NMR (100 MHz, CDCl₃) δ 165.4 (C), 150.8 (C), 126.4 (CH), 120.6 (q, J=320 Hz, CF₃), 92.7 (CH), 79.3 (CH₂O), 72.2 (CHN), 71.8 (CH₂O), 63.0 (CHN), 61.5 (CMe₂), 32.4 (CH), 31.0 (CH), 26.0 (2×CH₃), 18.5 (CH₃), 18.1 (CH₃), 17.6 (CH₃), 16.1 (CH₃); ¹⁹F NMR (300 MHz, CDCl₃) δ −78.6 (CF₃).

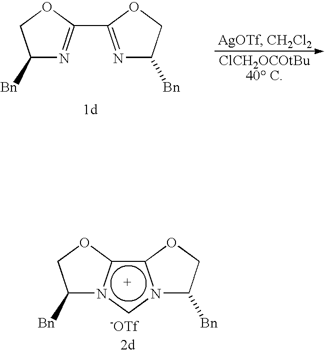

### Example 13

### Preparation of imidazolium triflate 2d

1.7 g, 36%, white foam; [a]²⁰_{D}=+38.1 (c 1.4, CH₂Cl₂); ¹H NMR (400 MHz, CDCl₃) δ 8.04 (s, 1H, NCHN), 7.31-7.20 (m, 10H, CHₐᵣ), 5.27-5.20 (m, 2H, CH), 5.00 (dd, J=7.5, 9.1 Hz, 2H, CH₂), 4.75 (dd, J=5.8, 9.0 Hz, 2H, CH₂), 3.40 (dd, J=6.2, 13.9 Hz, 2H, CH₂Ph), 3.10 (dd, J=8.1, 13.9 Hz, 2H, CH₂Ph); ¹³C NMR (100 MHz, CDCl₃) δ 133.8 (C), 129.2 (CH), 129.2 (CH), 127.9 (CHₐᵣ), 125.8 (C), 120.6 (q, J=321 Hz, CF₃), 115.2 (NCHN), 81.0 (OCH₂), 59.7 (NCH), 38.5 (CH₂) δ ¹⁹F NMR (300 MHz, CDCl₃) δ −78.5 (CF₃).

### Working Example for Claim 22

The Suzuki cross-coupling has become a standard synthetic method for biaryls in the academic and industrial sphere. (N. Miyaura, A. Suzuki, Chem. Rev. (1995) 95, 2457; S. Kotha, K. Lahiri, D. Kashinath, Tetrahedron (2002) 58, 9633.) While aryl iodides and bromides normally serve as substrates, the development of novel catalyst systems has recently also made it possible to efficiently couple the cheaper and more readily available aryl chlorides. (Review article: A. F. Littke, G. C. Fu, Angew. Chem. (2002) 114, 4350.) In spite of some effort, it has hitherto not been possible to couple aryl chlorides to biaryls having more than one ortho-substituent at room temperature. (Synthesis of sterically hindered biaryls by Suzuki cross-coupling at higher temperatures: J. P. Wolfe, R. A. Singer, B. H. Yang, S. L. Buchwald, J. Am. Chem. Soc. (1999) 121, 9550; A. F. Littke, C. Dai, G. C. Fu, J. Am. Chem. Soc. (2000) 122, 4020; J. Yin, M. P. Rainka, X.-X. Zhang, S. L. Buchwald, J. Am. Chem. Soc. (2002) 124, 1162.)

The use of a catalyst prepared from Pd(OAc)₂ and one equiv. of imidazolium salt 8 makes it possible to synthesize numerous biaryls from various aryl chlorides and arylboronic acids at room temperature. As shown in table 1, it is possible to couple unsubstituted, mono-ortho- and di-ortho-substituted aryl chlorides to a multitude of arylboronic acids in good to very good yields, with turnover numbers of up to 1730 at room temperature. In addition, it is possible to couple the sterically hindered 2,6-dimethylbenzeneboronic acid with unsubstituted, ortho-substituted, electron-deficient and -rich aryl chlorides (table 2). These results constitute the first Suzuki cross-couplings of aryl chlorides and arylboronic acids for the preparation of di- and triortho-substituted biaryls at room temperature.

TABLE 1
Suzuki coupling of sterically hindered aryl
chlorides.^{[a]}

| | Aryl | | | |
|---|---|---|---|---|
| No. | chloride | Boronic acid | Product | Yield^{[b]} |
| 1 | 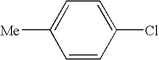 | 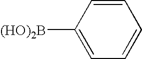 | 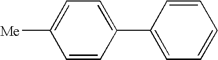 | 82%^{[c]} |
| 2 | 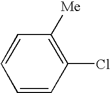 | 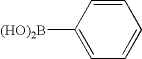 | 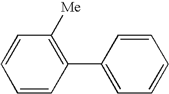 | 83% |
| 3 | 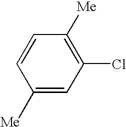 | 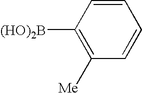 | 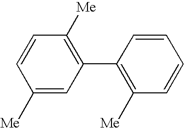 | 94% |
| 4 | 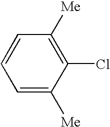 | 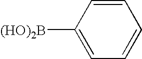 | 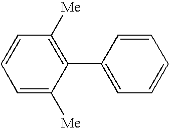 | 79%^{[c]} |
| 5 | ″ | 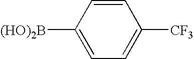 | 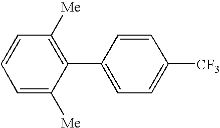 | 85% |
| 6 | ″ | 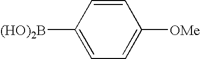 | 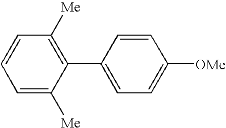 | 87% |
| 7 8 | 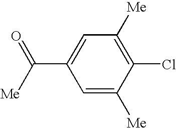 | 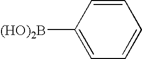 | 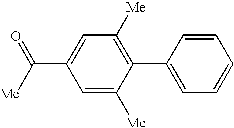 | 52%^{[d]}94%^{[e]} |

^{[a]}Reaction conditions: 3 mol % of Pd(OAc)₂, 1 (prepared from 3.1 mol % of 2, 6.25 mol % of KH, 0.67 mol % of KOtBu in THF); 1.0 equiv. of aryl chloride (1 mmol), 1.1 equiv. of boronic acid, 2.0 equiv. of CsF, THF [0.3 M], RT, 24 h. (Reaction times were not optimized).
^{[b]}Isolated yield.
^{[c]}<5% of product were obtained using 5 as a catalyst.
^{[d]}0.03 mol % of catalyst.
^{[e]}0.03 mol % of catalyst, at 60° C.

TABLE 2
Suzuki coupling of sterically hindered boronic
acids.^{[a]}

| | Aryl | | | |
|---|---|---|---|---|
| No. | chloride | Boronic acid | Product | Yield^{[b]} |
| 1 | 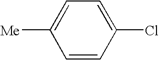 | 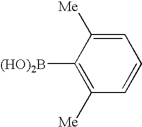 | 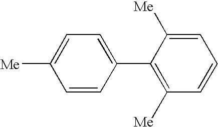 | 70% |
| 2 | 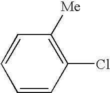 | ″ | 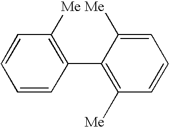 | 69% |
| 3 | 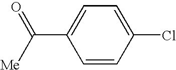 | ″ | 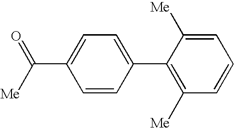 | 95% |
| 4 | 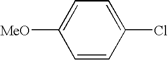 | ″ | 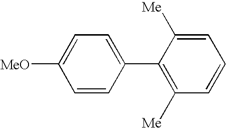 | 72% |
| 5 | 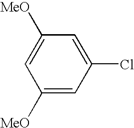 | ″ | 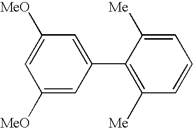 | 76% |

^{[a]}Reaction conditions: 3 mol % of Pd(OAc)₂, 1 (prepared from 3.1 mol % of 2, 6.25 mol % of KH, 0.67 mol % of KOtBu in THF); 1.0 equiv. of aryl chloride (1 mmol), 1.1 equiv. of boronic acid, 2.0 equiv. of KOtBu; THF/H₂O [10:1, 0.3 M], RT, 24 h (reaction times were not optimized).
^{[b]}Isolated yield.

### Comparative Example 1

The synthesis of the chloride of an imidazolium salt 6a was described in the literature with a yield of 40% after a reaction time of 5 days. (A. J. Arduengo, III, R. Krafczyk, R. Schmutzler, Tetrahedron (1999) 55, 14523.)

### Comparative Example 2

The synthesis of the chloride of an imidazolium salt 6b was described in the literature with a yield of 47% after a reaction time of 16 hours. (A. J. Arduengo, III, R. Krafczyk, R. Schmutzler, Tetrahedron (1999) 55, 14523.)

### Comparative Example 3

The attempted synthesis of the chloride of an imidazolium salt 2a, 2b and 2c by the method described in the literature (A. J. Arduengo, III, R. Krafczyk, R. Schmutzler, Tetrahedron (1999) 55, 14523.) for bisimines gave a complex mixture of several products.

1-22. (canceled)

23. A process for preparing an imidazolium salt of the formulae II, IV or VI, said process comprising reacting the corresponding substrate of the formulae I, III or V: 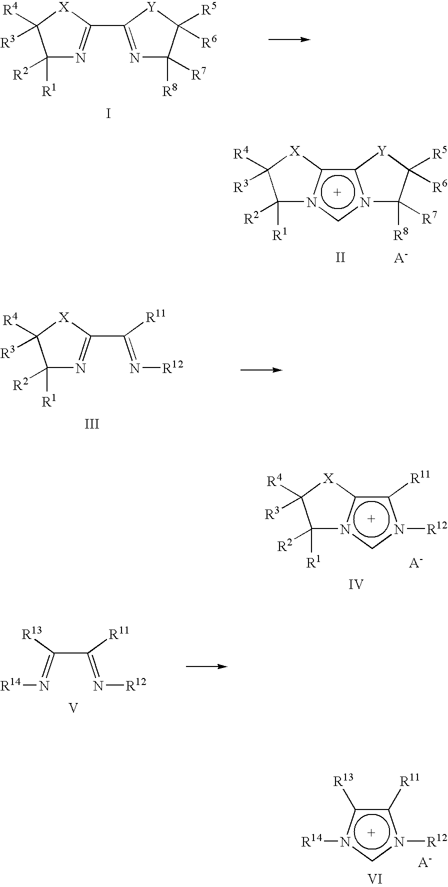
where
R¹, R², R³, R⁴, R⁵, R⁶, R⁷, R⁸, R⁹, R¹⁰, R¹¹, R¹², R¹³ and R¹⁴ are the same or different and are saturated or unsaturated, straight-chain, branched or cyclic, unsubstituted or substituted C₁₋₁₀-alkyl, C₂₋₅-alkenyl, C₂₋₅-alkynyl, C₇₋₁₉-aralkyl or C₆₋₁₄-aryl substituents, or R¹, R², R³, R⁴, R⁵, R⁶, R⁷, R⁸, R¹¹ and R¹³ may also be hydrogen or, together, form fused, substituted or unsubstituted substituents having 3-7 carbon atoms, R¹¹ and R¹³ may also be —OR¹⁶, —SR¹⁷ or —NR¹⁸R¹⁹, in which R¹⁶, R¹⁷, R¹⁸ and R¹⁹ may each be as defined for the R¹ to R⁸ and R¹¹ to R¹⁴ substituents, and R¹⁶, R¹⁷, R¹⁸, R¹⁹, one of the R¹, R², R⁷, R⁸, R¹² and R¹⁴ substituents may also be a linker L to a further imidazolium salt of the formula II, IV or VI,
X is O, S, an NR⁹ or CR^{9a}R^{9b} group in which R⁹, R^{9a} and R^{9b} are each hydrogen, saturated or unsaturated, straight-chain, branched or cyclic, unsubstituted or substituted C₁₋₁₀-alkyl, C₂₋₅-alkenyl, C₂₋₅-alkynyl, C₇₋₁₉-aralkyl or C₆₋₁₄-aryl substituents,
Y is O, S, an NR¹⁰ or NR^{10a}R^{10b} group in which R¹⁰, R^{10a}, R^{10b} are hydrogen, saturated or unsaturated, straight-chain, branched or cyclic, unsubstituted or substituted C₁₋₁₀-alkyl, C₂₋₅-alkenyl, C₂₋₅-alkynyl, C₇₋₁₉-aralkyl or C₆₋₁₄aryl substituents, and A⁻ is a mono- or polyvalent, organic or inorganic anion or a metal complex ion,
with a combination of an alkylating agent of the formula VII, VIII or IX: 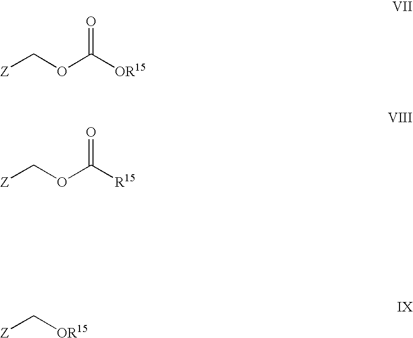
where Z is a leaving group and R¹⁵ is as defined for R³, and a metal salt of the formula:
MA
where M is a mono- or polyvalent metal cation, a tetraorganoammonium compound or a triorganosilyl group, and A is as defined above for A⁻, as a promoter of the reaction.

24. The process as claimed in claim 23, wherein R¹, R², R³, R⁴, R⁵, R⁶, R⁷, R⁸, R⁹, R^{9a}, R^{9b}, R¹⁰, R^{10a}, R¹¹, R¹², R¹³, R¹⁴ and R¹⁵ are the same or different and are each saturated or unsaturated, straight-chain, branched or cyclic, unsubstituted or substituted C₁₋₆-alkyl, C₂₋₄alkenyl, C₂₋₄-alkynyl, C₇₋₁₀-aralkyl or phenyl groups.

25. The process as claimed in claim 23, wherein the mono- or polyvalent, organic or inorganic anion A⁻ in the formulae II, IV and VI is a sulfate, halide, pseudohalide, borate, phosphate or metal complex ion or an optionally halogenated sulfonate, carboxylate or acetylacetonate ion.

26. The process as claimed in claim 25, wherein A⁻ is a triflate, mesylate, tosylate, nonaflate, tresylate, benzenesulfonate, brosylate, nosylate, fluorosulfonate, tetraphenylborate, tetrakis[3,5-bis(trifluoromethyl)phenyl]borate (BARF), tetrafluoro-borate, hexafluorophosphate, hexafluoroantimonate, acetate, trifluoroacetate, perchlorate, tetracarbonylcobaltate or hexafluoroferrate(III) ion.

27. The process as claimed in claim 26, wherein A⁻ in the formulae II, IV and VI is a triflate ion.

28. The process as claimed in claim 23, wherein R¹, R², R³, R⁴, R⁵, R⁶, R⁷, R⁸, R⁹, R^{9a}, R^{9b}, R¹⁰, R^{10a}, R^{10b}, R¹¹, R¹², R¹³, R¹⁴ and R¹⁵ are substituted by one or more, identical or different amine, nitro, nitrile, isonitrile, ether, alcohol, aldehyde or ketone groups, carboxylic acid derivatives, halogenated hydrocarbon groups, carbohydrate, phosphane, phosphane oxide, phosphane sulfide, phosphole groups, phosphite derivatives, aliphatic or aromatic sulfonic acid derivatives, the salts, esters or amides thereof, silyl functions, boryl groups or heterocyclic substituents.

29. The process as claimed in claim 28, wherein one of the R¹, R², R⁷, R⁸, R¹² and R¹⁴ groups is substituted by an azolium salt or a pyridine ring.

30. The process as claimed in claim 23, wherein the leaving group Z is a halide, pseudohalide or carboxylate.

31. The process as claimed in claim 30, which comprises using an alkylating agent of the formula VII, VIII or IX, where Z is a halide and R¹⁵ is an unsubstituted or substituted phenyl, benzyl or C₁-C₄-alkyl groups which may in each case contain one or more substituents.

32. The process as claimed in claim 31, wherein the alkylating agent used is chloromethyl pivalate, chloromethyl butyrate, chloromethyl ethyl ether, (2-methoxyethoxy)methyl chloride or (2-chloromethoxy-ethyl)trimethylsilane.

33. The process as claimed in claim 32, wherein the alkylating agent used is chloromethyl pivalate.

34. The process as claimed in claim 23, wherein the mono- or polyvalent metal cation M is a silver(I), alkali metal and alkaline earth metal, lanthanide, lead(II), mercury(II), cadmium(II), thallium(I), copper(II), zinc(II) or aluminum(III) ion, the tetraorganoammonium compound is a tetraalkylammonium compound and the triorganosilyl group is a trialkylsilyl group.

35. The process as claimed in claim 23, wherein a metal salt of the formula MA is used where M is silver(I) and A is a sulfate, halide, pseudohalide, borate, phosphate or metal complex ion or an optionally halogenated sulfonate, carboxylate or acetylacetonate ion.

36. The process as claimed in claim 35, wherein A is a triflate ion.

37. The process as claimed in claim 23, wherein one of the R¹, R², R⁷, R⁸, R¹² and R¹⁴ substituents is a linker L to a further imidazolium salt of the formula II, IV or VI.

38. The process as claimed in claim 37, wherein L is a C₁₋₄alkylene, C₅₋₁₂-cycloalkylene, C₆₋₁₂-arylene or C₆₋₁₂heteroarylene group which may optionally be substituted or be interrupted by a heteroatom or a cyclic substituents.

39. The process as claimed in claim 38, wherein the imidazolium salt has the general formula X:

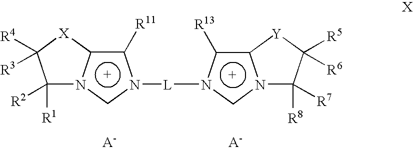

40. The process as claimed in claim 23, wherein the alkylating agent and metal salt are used in at least a stoichiometric amount based on the particular substrate.

41. The process as claimed in claim 23, which further comprises preparing beforehand one a reagent composed of the metal salt and the alkylating agent.

42. The process as claimed in claim 23, wherein the substrates are reacted in an organic solvent.

43. The process as claimed in claim 42, wherein the organic solvent is acetone, diethyl ether, methyl tertbutyl ether, petroleum ether, acetonitrile, propionitrile, ethyl acetate, benzene, toluene, xylene, benzine, 1,2-dichloroethane, chloroform or methylene chloride.

44. The process as claimed in claim 43, wherein the solvent is methylene chloride.

45. A compound of the general formula II, IV or XI: 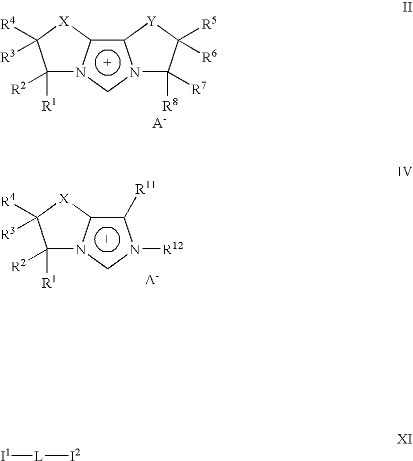
where
I¹ and I² are identical or different imidazolium salts of the formulae II, IV and VI which are joined to L at the position of the R¹, R², R⁷, R⁸, R¹² or R¹⁴ substituents, with the proviso that I¹ and I² are not both an imidazolium salt of the formulae VI, the imidazolium salt of the formula VI, R¹, R², R³, R⁴, R⁵, R⁶, R⁷, R⁸, R¹¹, R¹², R¹³, R¹⁴, X, Y, L and A⁻ are each as defined in claim 23.

46. A compound as claimed in claim 45, which has the following structural formulae: 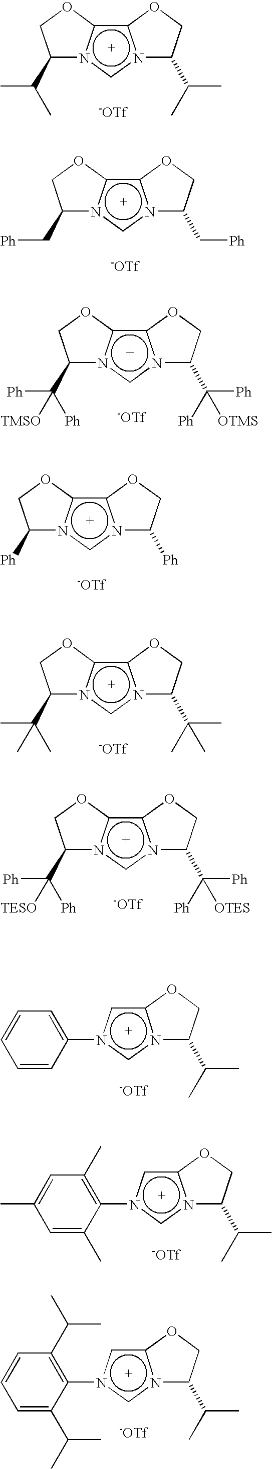 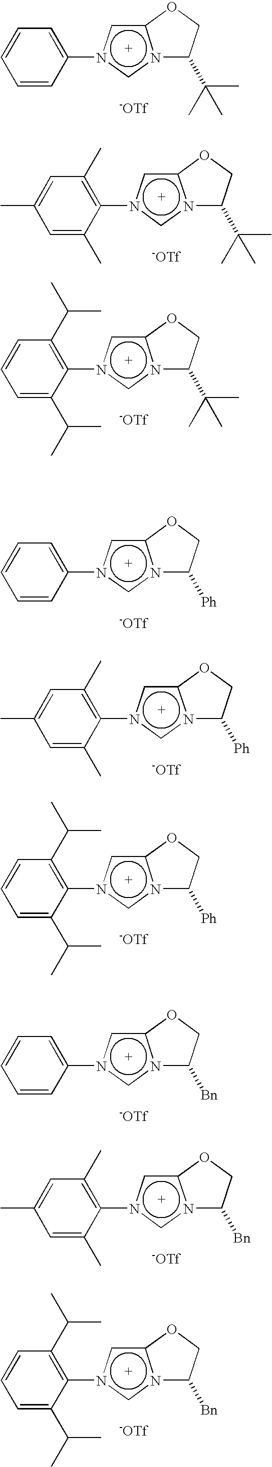
where OTf is trifluoromethanesulfonate (triflate), Ph is phenyl, TMS is trimethylsilyl, TES is triethylsilyl and Bn is benzyl.

47. A compound as claimed in claim 45, which is a compound of the formula X:

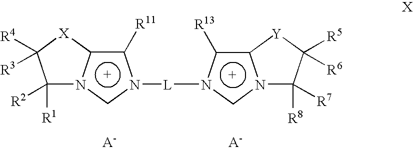

48. A compound as claimed in claim 47, which has the following structural formulae: 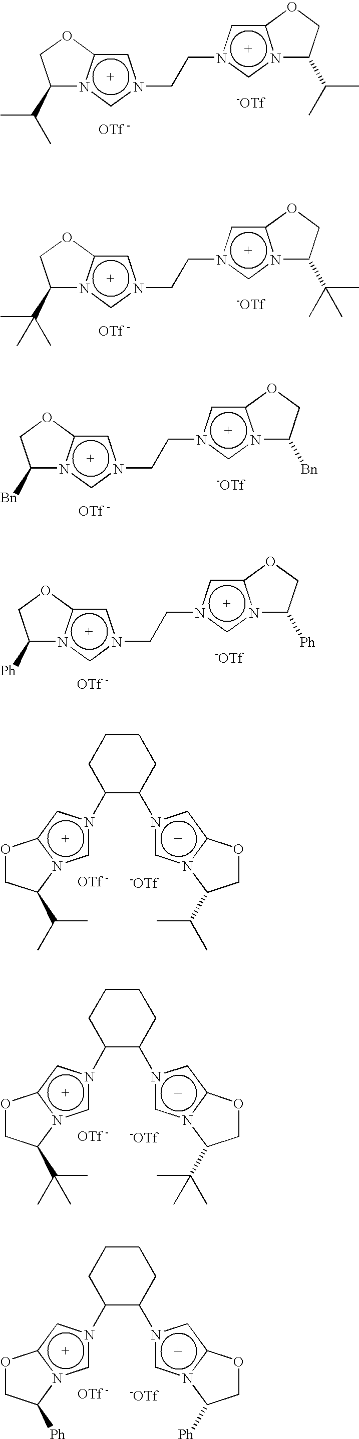 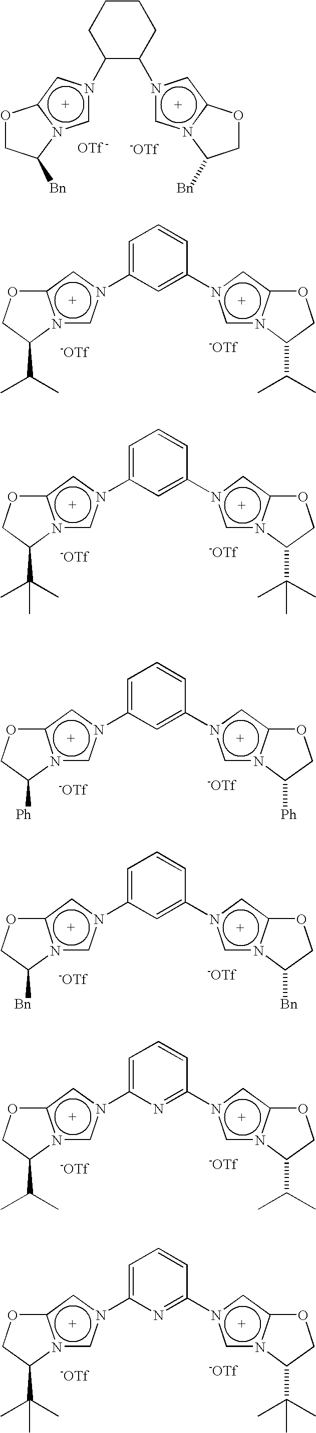 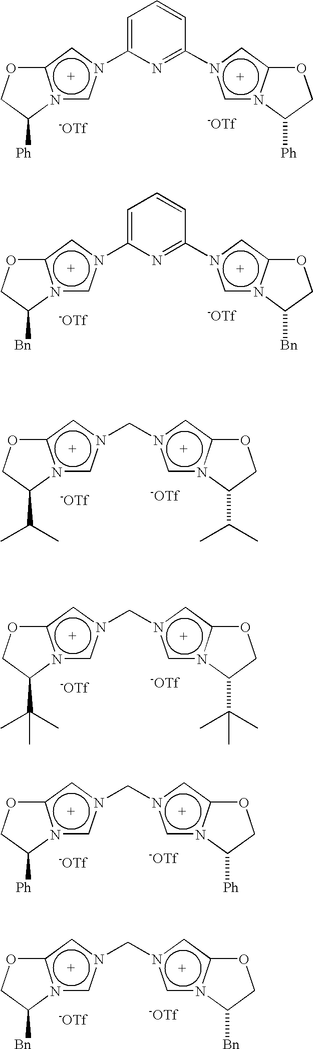
where OTf is trifluoromethanesulfonate (triflate), Ph is phenyl and Bn is benzyl.

49. A compound having the structure of the compound of claim 46, but having tetrafluoroborate, mesylate, tosylate, nonaflate or hexafluoroantimonate instead of triflate as the counteranion.

50. A compound having the structure of the compound of claim 48, but having tetrafluoroborate, mesylate, tosylate, nonaflate or hexafluoroantimonate instead of triflate as the counteranion.

51. A method for preparing a catalyst in the form of metal complexes of N-heterocyclic carbenes, said method comprising deprotonating a compound as claimed in claim 45.
